(19) United States
(12) Patent Application Publication
(10) Pub. No.: US 2010/0241094 A1
(43) Pub. Date: Sep. 23, 2010

(54) SMART DIAPER

(76) Inventor: Mark SHERRON, Harrisburg, PA (US)

Correspondence Address: MCNEES WALLACE & NURICK LLC, 100 PINE STREET, P.O. BOX 1166, HARRISBURG, PA, 17108-1166, US

(21) Appl. No.: 12/723,748

(22) Filed: Mar. 15, 2010

Related U.S. Application Data
(60) Provisional application No. 61/161,947, filed on Mar. 20, 2009.

(51) Int. Cl. A61F 13/45

(52) U.S. Cl. 604/361

(57) ABSTRACT

The invention is directed to an absorbent article which has an outer cover, an inner absorbent material and a sensing system. The sensing system has a measuring device, a monitoring area and a warning device. The monitoring area has a plurality of conductors which are in electrical engagement with the measuring device. The conductors of the monitoring area are spaced apart to sense if an insult has occurred and to sense the size of the insult. A warning device is electrically connected to the measuring device. The warning device provides information to the caregiver regarding the occurrence and size of the liquid or solid insult.

### FIELD OF THE INVENTION

The present invention is directed to a diaper which can sense whether the diaper has been soiled and which can signal a remote device when the diaper is soiled, and in particular to a diaper which sends different signals relative to the type of soiling and size of the soiled area.

### BACKGROUND OF THE INVENTION

There has long been a need for the detection of soiled diapers and remote notification of a caregiver. This is important in the case of babies who would develop diaper rash when left for long periods of time in a soiled diaper. It is also important in the case of incontinent adults unable to communicate with attendants. Adults are also susceptible to diaper rash, and in adults, this is far more serious condition.

In baby day care centers, diapers are usually changed at timed intervals, which does not solve the diaper rash problem, as a baby can soil his or her diaper soon after the diaper change. The baby must then remain in a soiled diaper until the next scheduled change. Of course, this problematical situation also applies to incontinent adults. In addition, there are many adults who are ambulatory but have lost bladder control and sensitivity in the groin area. Such adults realize they are soiled only after the diaper leaks, causing embarrassment.

Disposable diapers have contributed significantly towards enhancing and maintaining a healthy hygienic environment for the person wearing such a device. This is primarily because the absorbent fabrics, fibers and materials now found in many commercially available disposable diapers are very effective in sequestering waste products away from the wearer, thereby reducing the conditions conducive for developing diaper rash.

Although disposable diapers can contribute significantly towards improving the overall health and hygiene of the wearer, it is still prudent to remove a disposable diaper once it has been soiled.

An assortment of wetness detector systems and associated devices have been previously proposed and implemented to monitor the condition of diapers, adult incontinence briefs, and other similar articles. The general principle of many wetness detection systems is to implement some sort of urine or wetness detector in cooperation with a display or a warning device. Some systems activate audible or visible alarms to indicate the presence of urine within the diaper. This is typically accomplished by the detection of some threshold wetness level within the diaper.

Concerned parents or guardians have a desire to know whether or not their infant or an infirm adult has been subjected to excessive time in a wet diaper, for example, because of a dilatory caregiver. This information is often helpful to permit a parent or guardian to qualitatively assess care provided by a caregiver to an infant or infirm adult. Some systems purport to track and record the timing and frequency of urinating events and associated diaper changes. However, these systems only track whether an event has occurred; they do not track the volume of the event.

It would, therefore, be beneficial to provide a diaper which could sense whether the diaper has been soiled and to what extent the diaper has been soiled, and which could remotely alert the caregiver (parent, sitter, or health care worker) that the diaper requires changing. In addition, it would be beneficial to provide a monitoring system which would provide information to the parent or guardian as to the quality and timeliness of care given to the infant or infirm adult.

### SUMMARY OF THE INVENTION

One aspect of the invention is directed to an absorbent article which has an outer cover, an inner absorbent material and a sensing system. The sensing system has a measuring device, a monitoring area and a warning device. The monitoring area has a plurality of conductors which are in electrical engagement with the measuring device. The conductors of the monitoring area are spaced apart to sense if an insult has occurred and to sense the size of the insult. A warning device is electrically connected to the measuring device. The warning device provides information to the caregiver regarding the occurrence and size of the liquid or solid insult.

A power source may be provided and is electrically connected to the conductors. The power source provides power to the measuring device and the warning device.

The conductors may have a respective positive and negative charge provided thereon. When the absorbent article is dry, a circuit across the conductors is open, as an electrical measuring resistance develops between the conductors. When the absorbent article is soiled, the charge bridges the conductors, completing the circuit.

The monitoring area may include a plurality of conductor pairs, the conductor pairs separated by a preselected distance. The conductors in the monitoring area may have three conductor pairs, with a third conductor pair being spaced further apart than a second conductor pair and the second conductor pair being spaced apart further than a first conductor pair. The first conductor pair may have a longer length than the second conductor pair and the second conductor pair has a longer length than the third conductor pair. Alternatively, the conductors of the monitoring area form a grid.

The warning device may be an audible alarm which is activated when a circuit is completed between any respective conductor pairs. The audible alarm may vary depending upon the amount of conductors bridged and the size of the circuit completed. In the alternative, the warning device may be a signal transmitter which is activated when a circuit is completed between any respective conductors. A signal transmitted by the signal transmitter may vary depending upon the amount of conductors bridged and the size of the circuit completed.

Another aspect of the invention is directed to an absorbent article which has an outer cover, an inner absorbent material and a sensing system. The sensing system has a first monitoring area extending from proximate a front waist region of the absorbent article to proximate a crotch region, and a second monitoring area extending from proximate a back waist region to proximate the crotch region. Conductors positioned in the first monitoring area and the second monitoring area are in electrical engagement with a measuring device. The conductors of the first monitoring area are spaced apart to sense if a liquid insult has occurred and to sense the size of the liquid insult. The conductors of the second monitoring area are spaced apart to sense if a solid insult has occurred and sense the size of the solid insult. A warning device is electrically connected to the measuring device and provides information to the caregiver regarding the occurrence and size of the liquid or solid insult.

Another aspect of the invention is directed an absorbent article which has an outer cover, an inner absorbent material and a sensing system. The sensing system has a first monitoring area which extends from proximate the front waist region of the absorbent article to proximate the crotch region, and a second monitoring area which extends from proximate the back waist region to proximate the crotch region. A measuring device is provided for determining whether a liquid insult has occurred in the first monitoring area and the size of the liquid insult. The measuring device also determines whether a solid insult has occurred in the second monitoring area and the size of the solid insult. A warning device is electrically connected to the measuring device. The warning device provides information to the caregiver regarding the occurrence and size of the liquid or solid insult.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 is a top view of a first embodiment of a smart diaper according to the present invention, having a first and a second monitoring area with discrete conductors extending therein.

FIG. 2 is a top view of a second embodiment of the smart diaper according to the present invention, having a first and a second monitoring area with circuit grids positioned therein.

### DETAILED DESCRIPTION OF THE INVENTION

The embodiments disclosed are directed to an absorbent article 2 and to a sensing or monitoring system 4 for detecting a need to change the absorbent article 2 when soiled. The drawings and descriptions describe an absorbent article 2 in the form of a diaper. The absorbent article 2 may or may not be disposable, which refers to articles that are intended to be discarded after a limited period of use instead of being laundered or otherwise conditioned for reuse. It is understood that the present invention is suitable for use with various other absorbent articles 2 intended for personal wear, including but not limited to incontinence products, medical garments, other personal care or health care garments, and the like without departing from the scope of the present invention.

Although not required, the absorbent article 2 may have an inner panel, an outer panel, and an absorbent material or pad (not shown). The outer or cover panel may be attached to the inner panel. The inner panel may be configured to be liquid pervious, while the outer panel may be configured to be substantially liquid impervious. The absorbent pad may be interposed between the inner and outer panels.

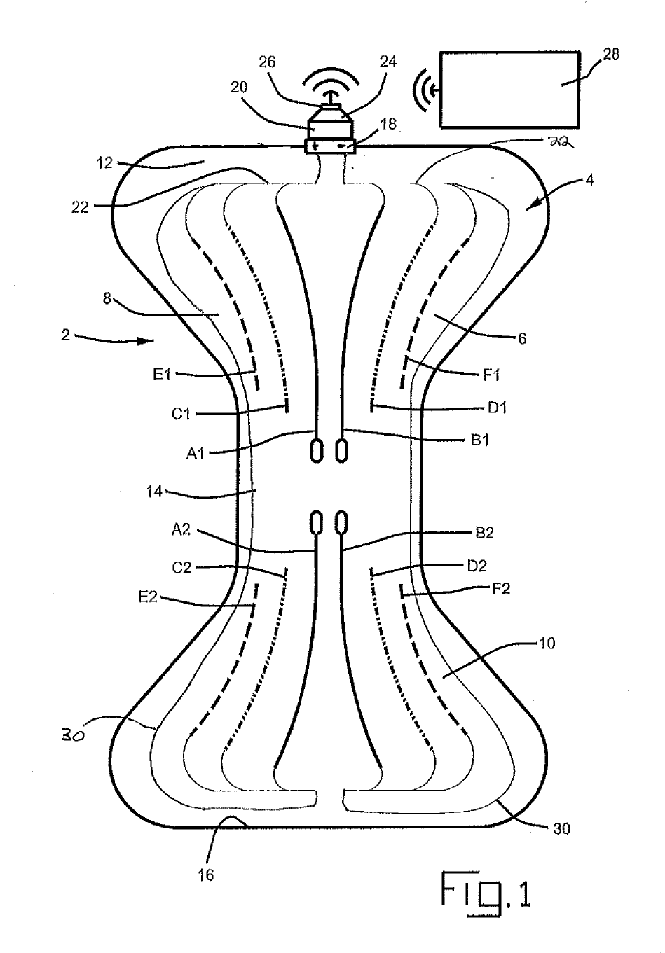

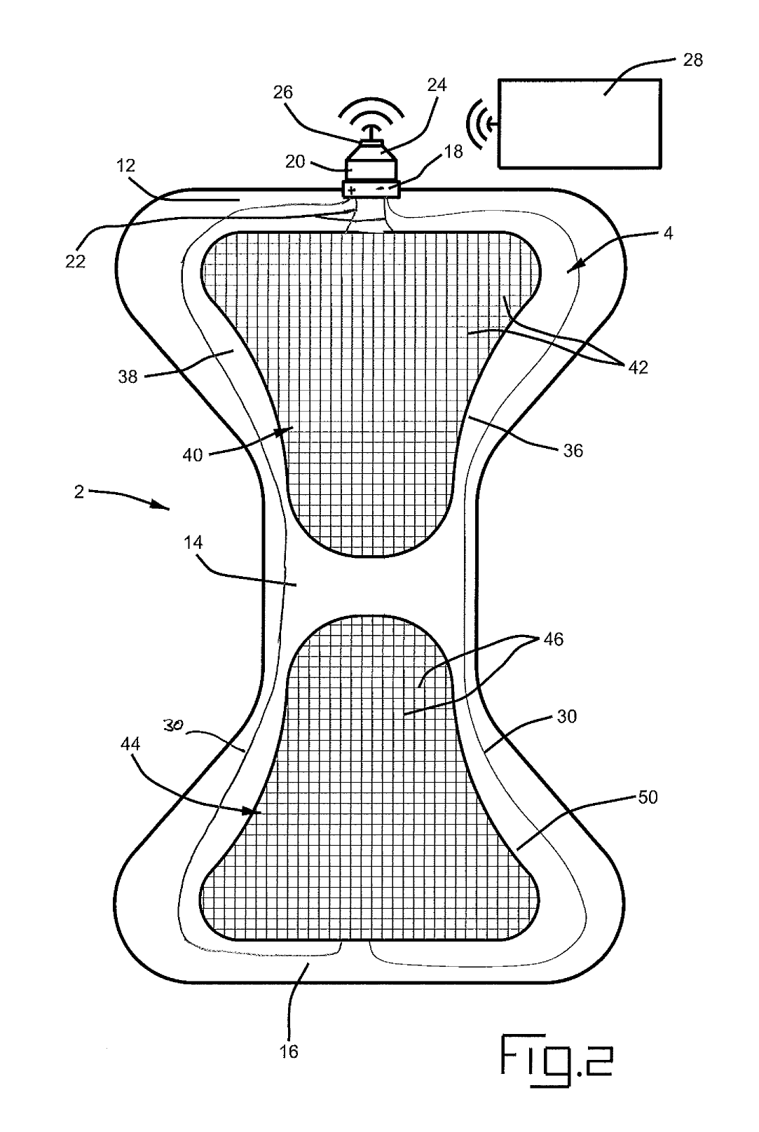

Regardless of the number of panels and layers, the monitoring system 4 includes at least one insult sensor 6, 36 for detecting the electrical property (e.g., resistance, conductance, capacitance, inductance) of the article. The insult sensor 6 may include a plurality of spaced-apart conductors disposed within the diaper, as shown in FIG. 1, or may include a plurality of spaced-apart conductors disposed in a grid within the diaper, as shown in FIG. 2. In either embodiment, a first set of conductors define a first monitoring area 8 of the diaper 2 and a second set of conductors define a second monitoring area 10 of the diaper 2. The conductors may be constructed of any material that is generally electrically conductive. For example, the conductors may be constructed of metal strips, metal or conductive wire, metal films, coated films, conductive polymers, conductive inks, or conductive threads. Other conductors are within the scope of this invention. The first set of conductors extend longitudinally from proximate a front waist region 12 to proximate a crotch region 14, and the second set of conductors extend longitudinally from proximate a back waist region 16 to proximate the crotch region 12. The conductors may be disposed within the absorbent assembly between the outer layer and the absorbent pad, or the conductors may be disposed at other locations without departing from the scope of this invention.

Regardless of the embodiment, current from a current or power source 18 runs through the conductors of the sensor 6. The current source 18 may be a single current source, a plurality of current sources, or a combination of current sources and current accumulators. The current source may be a direct current source, an alternating current source or a high capacity capacitor. The current source may provide electrical energy for the sensing system from ambient energy. The conductors may be electrically connected to the current source 18 by way of electrically conductive snap fasteners (not shown). Other ways of electrically connecting the conductors to the current source 18 are within the scope of this invention. A housing, which carries the current source and may carry other components, may be releasably secured to the diaper 2 by way of the snap fasteners, although it is understood that the housing may be releasably secured using other means or may be permanently secured to the diaper 2 without departing from the scope of this invention.

Because the conductors are spaced apart, current from the current source 18 must pass through the monitoring areas 8, 10 to complete the circuit. Each monitoring area 8, 10 acts essentially as a resistor. When the monitoring areas 8, 10 are dry (e.g., before soiling or the presence of an insult), the resistance of the monitoring area is relatively high. When the monitoring area is wetted or soiled, its resistance drops, because of the electrically conductive nature of the insult.

A measuring device or controller 20, e.g. an application-specific integrated circuit (ASIC), is positioned in electrical engagement with the first monitoring area 8 of the diaper to measure the electrical properties of the first monitoring area 8. A separate measuring device or controller or a separate circuit of the same measuring device 20 also measures the electrical properties of the second monitoring area 10 of the diaper 2. The measuring device 20 may be configured with appropriate memory for processing, recoding and storing data received from the insult sensor, as will be more fully described.

Referring to the first sensor 6 shown in the first disclosed embodiment of FIG. 1, the first monitoring area 8 is used to monitor whether a liquid insult has occurred. The first monitoring area 8 has a first circuit which has a conductor A₁ and a conductor B₁, a second circuit which has a conductor C₁ and a conductor D₁, the conductors C₁ and D₁ being spaced apart further than the conductors A₁ and B₁, and a third circuit which has a conductor E₁ and a conductor F₁, the conductors E₁ and F₁ being spaced apart further than the conductors C₁ and D₁. All of the conductors are connected to the power source 18 via a power conductor 22 which is positioned at a respective end of each of the conductors A₁, B₁, C₁, D₁, E₁ and F₁.

When the diaper 2 is in use and the wearer soils the diaper 2 or produces an insult, the liquid insult is absorbed by the diaper 2. When the liquid is disseminated in the diaper 2 and the insult reaches the conductors A₁ and B₁, an alarm device or component 24 or signal transmitter 26 is activated to indicate that the liquid in the diaper 2 has reached the respective conductors. The alarm device and the signal transmitter are individually or in combination referred to as a warning device. This occurs because the conductors act as a bridge circuit. The conductors, which are electrically connected to the power source 18, have a respective positive and negative charge provided thereon. When the diaper is dry, the circuit across the conductors is open as an electrical measuring resistance develops between the conductors. Since the dry diaper is not a good conductor of electricity, the charge on the conductors does not bridge across the conductors. However, the liquid insult has a different electrical resistance and conducts electricity much better. Therefore, the introduction of liquid insult between the conductors allows the charge to bridge the conductors, completing the circuit. When the circuit is complete between conductors A₁ and B₁, the alarm device 24 and/or the signal transmitter 26 is activated to generate a first alarm to indicate that the liquid insult is present. An insult which is confined between conductors A₁ and B₁ is not considered large. As the amount of liquid with such an insult is not large, the diaper 2 may be able to effectively remove the liquid from the wearer's skin, thereby making it less critical for the caregiver to instantly change the diaper.

The use of the alarm device 24 provides notification of the liquid insult to a caregiver who may be physically located proximate the diaper 2. The use of the signal transmitter 26 and signal receiver 28 can provide notification to caregivers who are outside the range of an audible alarm. This allows the caregiver the freedom to conduct other tasks while effectively monitoring the presence of an insult. The use of signal transmitters 26 and receivers 28 also allows caregivers or other parties to monitor the effectiveness and timeliness of the caregiver. A log can be created which can be reviewed periodically to determine if proper attention is given to the wearer of the diaper 2.

The larger the insult, the more beneficial to change the diaper 2 more quickly. Therefore, when the liquid insult is disseminated in the diaper 2 and the insult reaches the conductors C₁ and D₁, the alarm device 24 is activated and produces a second, different alarm to indicate that the liquid insult in the diaper 2 has reached conductors C₁ and D₁. This occurs because the conductors act as a bridge circuit. As the concept of a bridge circuit has been previously described, the explanation will not be repeated. When the circuit is complete between conductors C₁ and D₁, the alarm device 24 and/or the signal transmitter 26 is activated to indicate that the liquid insult has reached the second circuit. In the alternative, the second alarm may be activated if the liquid insult has bridged conductors A₁ and B₁ and has reached either conductor C₁ or D₁.

As previously stated, if an insult is large, it is generally beneficial to change the diaper 2 more quickly. Therefore, when the liquid insult is disseminated in the diaper 2 and the insult reaches the conductors E₁ and F₁, the alarm device 24 is activated and produces a third, different alarm to indicate that the liquid insult in the diaper has reached conductors E₁ and F₁. This occurs because the conductors act as a bridge circuit. As the concept of a bridge circuit has been previously described, the explanation will not be repeated. When the circuit is complete between conductors E₁ and F₁, the alarm device 24 and/or the signal transmitter 26 is activated to indicate that the liquid insult has reached the third circuit. In the alternative, the third alarm may be activated if the liquid insult has bridged conductors A₁ and B₁ and has reached either conductor E₁ or F₁.

The second monitoring area 10 is used to monitor whether a solid insult has occurred. The second monitoring area 10 has a first circuit which has a conductor A₂ and a conductor B₂, a second circuit which has a conductor C₂ and a conductor D₂, the conductors C₂ and D₂ being spaced apart further than the conductors A₂ and B₂, and a third circuit which has a conductor E₂ and a conductor F₂, the conductors E₂ and F₂ being spaced apart further than the conductors C₂ and D₂. All of the conductors are connected to the power source 18 via a power conductor 30 which is positioned at a respective end of each of the conductors A₂, B₂, C₂, D₂, E₂ and F₂.

When the diaper 2 is in use and the wearer soils the diaper or produces a solid insult, the solid insult has a liquid component which is absorbed by the diaper 2. The liquid component of the solid insult also has a different electrical resistance and conducts electricity much better than the material of the diaper 2. Therefore, the introduction of solid insult between the conductors allows the charge to bridge the conductors, completing the circuit. When the insult reaches the conductors A₂ and B₂, an alarm device 24 and/or signal transmitter 26 is activated to indicate that the solid insult in the diaper 2 has reached the respective conductors. This occurs because the conductors act as a bridge circuit, as was previously described. When the circuit is complete between conductors A₂ and B₂, the alarm device 24 and/or the signal transmitter 26 is activated to indicate that the solid insult is present. A fourth alarm is used for a solid insult, different than that used for a liquid insult, thereby allowing the caregiver to distinguish between the two.

The use of the alarm device 24 provides notification of the solid insult to a caregiver who is physically located proximate the diaper 2. The use of such signal transmitters 26 and signal receivers 28 can provide notification to caregivers who are outside the range of an audible alarm. This allows the caregiver the freedom to conduct other tasks while effectively monitoring the presence of an insult. The use of signal transmitters 26 and receivers 28 also allows caregivers or other parties to monitor the effectiveness and timeliness of the caregiver. A log can be created which can be reviewed periodically to determine if proper attention is given to the wearer of the diaper 2.

As previously stated, the larger the insult, the more beneficial to change the diaper 2 more quickly. Therefore, when the solid insult reaches the conductors C₂ and D₂, the alarm device 24 and/or signal transmitter 26 is activated and produces a fifth, different alarm to indicate that the solid insult in the diaper 2 has reached conductors C₂ and D₂. This occurs because the conductors act as a bridge circuit. As the concept of a bridge circuit has been previously described, the explanation will not be repeated. When the circuit is complete between conductors C₂ and D₂, the alarm device 24 and/or the signal transmitter 26 is activated to indicate that the liquid insult has reached the second circuit. In the alternative, the fifth alarm may be activated if the solid insult has bridged conductors A₂ and B₂ and has reached either conductor C₂ or D₂.

As previously stated, if an insult is large, it is generally beneficial to change the diaper 2 more quickly. Therefore, when the solid insult reaches the conductors E₂ and F₂, the alarm device 24 and/or signal transmitter 26 is activated and produces a sixth, different alarm to indicate that the solid insult in the diaper has reached conductors E₂ and F₂. This occurs because the conductors act as a bridge circuit. As the concept of a bridge circuit has been previously described, the explanation will not be repeated. When the circuit is complete between conductors E₂ and F₂, the alarm device 24 and/or the signal transmitter 26 is activated to indicate that the solid insult has reached the third circuit. In the alternative, the sixth alarm may be activated if the solid insult has bridged conductors A₂ and B₂ and has reached either conductor E₂ or F₂.

The first monitoring area 8 and the second monitoring area 10 may be positioned in various layers of the diaper 2. As an example, the monitoring areas may be attached to the outer panel or to the absorbent pad.

In the embodiment shown, in the first monitoring area 8, the conductors A₁ and B₁ are longer than conductors C₁ and D₁, and the conductors C₁ and D₁ are longer than the conductors E₁ and F₁. This allows the ends of the conductors A₁ and B₁ to be positioned closer to the crotch, with the conductors C₁ and D₁ and conductors E₁ and F₁ positioned back therefrom in a stepped configuration. The ends of the conductors form a general V-shape.

While other configurations are possible, in the embodiment shown in FIG. 1, the second monitoring area 10 is essentially a mirror image of the first monitoring area. The conductors A₂ and B₂ are longer than conductors C₂ and D₂, and the conductors C₂ and D₂ are longer than the conductors E₂ and F₂. This allows the ends of the conductors A₂ and B₂ to be positioned closer to the crotch, with the conductors C₂ and D₂ and conductors E₂ and F₂ positioned back therefrom in a stepped configuration. The ends of the conductors form a general inverted V-shape.

The positioning of the conductors as described allows a more accurate reading of the size of the insult and minimizes false readings. The size of the insult must be large before conductors C₁, D₁, E₁ or F₁ or conductors C₂, D₂, E₂ or F₂ are bridged.

While the first sensor 6 has been described as measuring resistance, other embodiments are possible without departing from the scope of the invention. For example, an insult may be detected through capacitive rather than resistive coupling. In this embodiment, each conductor would be a pair of conductors which are interleaved to form what is essentially a parallel plate capacitor. Thus, each conductor of the pair of conductors is capacitively coupled to the other conductor of the pair of conductors. When the diaper is not soiled, the conductors of the pair of conductors are only weakly capacitively coupled. However, when the diaper is soiled, the capacitive coupling of the respective conductor pairs proximate the insult is increased, as the insult acts as an electrolyte. This increased capacitive coupling is sensed by the measuring device and causes the alarm or signal transmitter to be activated in the manner previously described.

Referring to the second sensor 36 shown in the second disclosed embodiment of FIG. 2, the first grid monitoring area 38 is used to monitor whether a liquid insult has occurred. The first grid monitoring area 38 has a first circuit grid 40. When the diaper 2 is in use and the wearer soils the diaper 2 or produces an insult, the liquid insult is absorbed by the diaper 2. The liquid is disseminated in the diaper 2 and the insult reaches the conductors 42 of the first circuit grid 40. Due to the configuration of the first circuit grid 40, the first circuit grid 40 can monitor and provide information with respect to the volume and size of the insult. Due to the close proximity of the conductors 42 of the grid 40, the first circuit grid 40 can provide detailed information regarding the volume of the insult. By counting the number of grids in which the resistance has changed, the measuring device or controller 20 can quickly and accurately determine the volume of the liquid insult. The use of a grid 40, as shown in FIG. 2, prevents a false positive from causing improper information to be conveyed to the parent or caregiver, as the measuring device 20 will accurately interpret the signals received from the first circuit grid 40. For instance, if a small stream of the insult has bridged a large width of the diaper 2, only select grids will record a resistance drop, allowing the measuring device 20 to determine that the volume of the insult is small, even though the width of the insult was large.

When an appropriate number of grids record a drop in resistance, or are otherwise activated, the alarm device 24 and/or signal transmitter 26 is activated to indicate that the liquid in the diaper 2 has reached the respective grids. This occurs because the conductors 42 act as a bridge circuit. The conductors 42, which are electrically connected to the power source 22, have a respective positive and negative charge provided thereon. When the diaper 2 is dry, the circuit across the conductors 42 is open as an electrical measuring resistance develops between the conductors 42. Since the dry diaper is not a good conductor of electricity, the charge on the conductors 42 does not bridge across the conductors 42. However, the liquid insult has a different electrical resistance and conducts electricity much better. Therefore, the introduction of liquid insult between the conductors 42 allows the charge to bridge the conductors 42, completing the circuit. When the circuit is complete between respective grids, the alarm device 24 and/or the signal transmitter 26 is activated to generate a first alarm to indicate that the liquid insult is present. An insult which is confined between the range of X and Y grids is not considered large. As the amount of liquid with such an insult is not large, the diaper 2 may be able to effectively remove the liquid from the wearer's skin, thereby making it less critical for the caregiver to instantly change the diaper. The number of grids which define X and Y can be programmed into the measuring device 20 and may be based on various factors, including the size of the grids.

The use of the alarm device 20 provides notification of the liquid insult to a caregiver who is physically located proximate the diaper 2. The use of signal transmitters 26 and receivers 28 can provide notification to caregivers who are outside the range of an audible alarm. This allows the caregiver the freedom to conduct other tasks while effectively monitoring the presence of an insult. The use of signal transmitters 26 and receivers 28 also allows caregivers or other parties to monitor the effectiveness and timeliness of the caregiver. A log can be created which can be reviewed periodically to determine if proper attention is given to the wearer of the diaper 2.

The larger the insult, the more beneficial to change the diaper 2 more quickly. Therefore, when the liquid insult is disseminated in the diaper 2 and the insult reaches the Y+1 to Z number of grids, the alarm device 24 and/or signal transmitter 26 is activated and produces a second, different alarm. This occurs because the conductors 42 act as a bridge circuit. As the concept of a bridge circuit has been previously described, the explanation will not be repeated. When the circuit is complete for Y+1 to Z number of grids, the alarm device 24 and/or the signal transmitter 26 is activated to indicate that the volume of the liquid has increased.

As previously stated, if an insult is large, it is generally beneficial to change the diaper 2 more quickly. Therefore, when the liquid insult is disseminated in the diaper 2 and the insult reaches Z+1 number of grids, the alarm device 24 and/or signal transmitter 26 is activated and produces a third, different alarm. This occurs because the conductors 42 act as a bridge circuit. As the concept of a bridge circuit has been previously described, the explanation will not be repeated. When the circuit is complete for Z+1 number of grids, the alarm device 24 and/or the signal transmitter 26 is activated to indicate that the liquid insult is large.

The amount of alarms and the number of grids that are bridged to cause the alarm to be activated for the first circuit grid 40 can be varied according to need. In order to vary the parameters, the measuring device 20 must be programmed accordingly.

The second grid monitoring area 50 is used to monitor whether a solid insult has occurred. The second grid monitoring area 50 has a second circuit grid 44. When the diaper 2 is in use and the wearer soils the diaper 2 or produces a solid insult, the solid insult has a liquid component which is absorbed by the diaper 2. The liquid component of the solid insult also has a different electrical resistance and conducts electricity much better than the material of the diaper. Therefore, the introduction of solid insult between the conductors 46 allows the charge to bridge the conductors, completing the circuit. Due to the configuration of the second circuit grid 44, the second circuit grid 44 can monitor and provide information with respect to the volume and size of the insult. Due to the close proximity of the conductors 46 of the grid 44, the second circuit grid 44 can provide detailed information regarding the volume of the insult. By counting the number of grids in which the resistance has changed, the measuring device 20 can quickly and accurately determine the volume of the solid insult

When an appropriate number of grids are activated, an alarm device 24 and/or signal component 26 is activated to indicate that the solid insult in the diaper 2 has reached the respective grids. This occurs because the conductors 46 act as a bridge circuit, as was previously described. A solid insult which is confined between the range of A and B number of grids is not considered large. The number of grids which define A and B can be programmed into the measuring device 20 and may be based on various factors, including the size of the grids. When the circuit is complete for between A and B number of grids, the alarm device 24 and/or the signal transmitter 26 is activated to indicate that the solid insult is present. A fourth alarm is used for a solid insult, different than that used for a liquid insult, thereby allowing the caregiver to distinguish between the two.

The use of the alarm device 24 provides notification of the solid insult to a caregiver who is physically located proximate the diaper. The use of signal transmitters 26 and receivers 28 can provide notification to caregivers who are outside the range of an audible alarm. This allows the caregiver the freedom to conduct other tasks while effectively monitoring the presence of an insult. The use of signal transmitters 26 and receivers 28 also allows caregivers or other parties to monitor the effectiveness and timeliness of the caregiver. A log can be created which can be reviewed periodically to determine if proper attention is given to the wearer of the diaper 2.

The larger the insult, the more beneficial to change the diaper 2 more quickly. Therefore, when the solid insult is disseminated in the diaper 2 and the insult reaches the B+1 to C number of grids, the alarm device 24 and/or signal transmitter 26 is activated and produces a fifth, different alarm. This occurs because the conductors 46 act as a bridge circuit. As the concept of a bridge circuit has been previously described, the explanation will not be repeated. When the circuit is complete for B+1 to C number of grids, the alarm device 24 and/or the signal transmitter 26 is activated to indicate that the volume of the solid has increased.

As previously stated, if an insult is large, it is generally beneficial to change the diaper 2 more quickly. Therefore, when the solid insult is disseminated in the diaper 2 and the insult reaches C+1 number of grids, the alarm device 24 and/or signal transmitter 26 is activated and produces a sixth, different alarm. This occurs because the conductors act as a bridge circuit. As the concept of a bridge circuit has been previously described, the explanation will not be repeated. When the circuit is complete for C+1 number of grids, the alarm device 24 and/or the signal transmitter 26 is activated to indicate that the solid insult is large.

Regardless of the size of the insult, the measuring device is able to determine by the signals sent by the second grid circuit 44 of the second monitoring area 50 if the insult is totally solid or if the insult has a liquid component, i.e. diarrhea. As the resistance is different between solids and liquids, the measuring device 20 is able to determine the concentration of liquid present.

The amount of alarms and the number of grids that are bridged to cause the alarm to be activated for the second circuit grid can be varied according to need. In order to vary the parameters, the measuring device 20 must be programmed accordingly.

While the second sensor 36 has been described as measuring resistance, other embodiments are possible without departing from the scope of the invention. For example, an insult may be detected through capacitive rather than resistive coupling. In this embodiment, each conductor would be a pair of conductors which are interleaved to form what is essentially a parallel plate capacitor. Thus, each conductor of the pair of conductors is capacitively coupled to the other conductor of the pair of conductors. When the diaper is not soiled, the conductors of the pair of conductors are only weakly capacitively coupled. However, when the diaper is soiled, the capacitive coupling of the respective conductor pairs proximate the insult is increased, as the insult acts as an electrolyte. This increased capacitive coupling is sensed by the measuring device and causes the alarm or signal transmitter to be activated in the manner previously described.

As previously stated with respect to FIG. 1, the first monitoring area 38 and the second monitoring area 50 of the second sensor 36 may be positioned in various layers of the diaper. As an example, the monitoring areas may be attached to the outer panel or attached to the absorbent pad.

In another embodiment 6, the first sensor, shown in FIG. 1, and the second sensor 36, shown in FIG. 2, may be combined into one diaper 2. In this embodiment, the first grid monitoring area 38, in conjunction with the first monitoring area 8, is used to monitor whether a liquid insult has occurred. The first grid monitoring area 38 has the first circuit grid 40. When the diaper 2 is in use and the wearer soils the diaper or produces an insult, the liquid insult is absorbed by the diaper 2. The liquid is disseminated in the diaper 2 and the insult reaches the conductors as described relative to FIG. 1. In addition, the liquid reaches the first circuit grid 40. Due to the configuration of the first circuit grid 40, the first circuit grid 40 can monitor and provide information with respect to the volume and size of the insult, as described relative to FIG. 2. In this embodiment, the measuring device 20 compares the information received from the conductors A₁, B₁, C₁, D₁, E₁, F₁ and the first circuit grid 40 to verify the size of the insult. If the information received from the first conductors and the first circuit grid 40 is consistent, an alarm device 24 and/or signal transmitter 26 is activated to indicate that the liquid in the diaper has reached the respective conductors, as previously described. If the conductors and the first circuit grid 40 provide inconsistent information (e.g. the conductors register a large insult and the first circuit grid 40 registers a small insult), the first circuit grid 40 information is used to determine which alarm is activated. This prevents a false positive from causing improper information to be conveyed (i.e. a small stream of the insult has caused conductor E₁ or F₁ to be bridged but the volume of the insult is still considered small).

The second grid monitoring area 50, in conjunction with the second monitoring area 10, is used to monitor whether a solid insult has occurred. The second grid monitoring area 50 has the second circuit grid 44. When the diaper 2 is in use and the wearer soils the diaper 2 or produces a solid insult, the solid insult has a liquid component which is absorbed by the diaper. The solid insult also has a different electrical resistance and conducts electricity much better than the material of the diaper 2. The solid insult reaches the conductors as described relative to FIG. 1. In addition, the solid insult reaches the second circuit grid 44. Due to the configuration of the second circuit grid 44, the second circuit grid 44 can monitor and provide information with respect to the volume and size of the solid insult, as described relative to FIG. 2. In this embodiment, the measuring device 20 compares the information received from the second conductors A₂, B₂, C₂, D₂, E₂, F₂ and the second circuit grid 44 to verify the size of the insult. If the information received from the second conductors and the second circuit grid 44 is consistent, an alarm device 24 and/or signal transmitter 26 is activated to indicate that the insult in the diaper 2 has reached the respective conductors, as previously described. If the second conductors and the second circuit grid 44 provide inconsistent information (e.g. the second conductors register a large insult and the second circuit grid 44 registers a small insult), the second circuit grid 44 information is used to determine which alarm is activated. This prevents a false positive from causing improper information to be conveyed (i.e. a small portion of the insult has caused conductor E₂ or F₂ to be bridged but the volume of the insult is still considered small).

When used in combination, the first sensor 6 and second sensor 36 may be provided in different layers in the diaper or may be combined into a single layer. One advantage of using both the first sensor 6 and the second sensor 36 in the same diaper, is that the measuring device 20 may be simplified, requiring less sophisticated electronics. The measuring device 20 is not required to interpret as much information; it is merely required to compare and respond to information received.

In each of the embodiments, the information transmitted by the insult sensors 6, 36 and collected and interpreted by the measuring device 20 may be stored and/or may be communicated to a display or receiving device which can provide the parent or caregiver with a history of the insults. The communication between the measuring device 20 and the receiving device 28 may be accomplished by radio frequency (RF) signals, infrared (IR) signals or in any other known manner.

A timer (not shown) may be embedded in the measuring device 20 to allow the information transmitted by the insult sensors 6, 36 to be viewed with respect to real and elapsed time. The measuring device 20 will record the time that an insult occurs (as discussed above) and the time when the diaper 2 is changed, thereby providing the elapsed time between the occurrence of the insult and the diaper change. The tracking of this information allows the parent or caregiver to determine the amount of time that the infant, toddler or diaper wearer remains in a soiled diaper 2. If the parent has placed the infant or toddler in the care of a caregiver, the parent can review how quickly the infant's or toddler's diaper 2 was changed and how long the infant or toddler remained in a soiled diaper 2. This will help the parent to determine whether the caregiver is providing adequate service.

The tracking of the number of insults over time also allows the parent or caregiver to determine if the child or diaper wearer is becoming dehydrated. If frequent insults occur over a short period of time, particularly if diarrhea is present, the measuring device may be programmed to activate an alarm to warn the parent or caretaker that the child is at risk for dehydration.

In any of the embodiments, the current or power source 18 may be positioned anywhere along the diaper 2, e.g. attached to the outer panel or attached to the absorbent pad. The current source 18 may be any commercially available source such as those selected from the group consisting of a battery and a high capacity capacitor. When the current source is in the form of a battery, it may be selected from the group consisting of a zinc-carbon battery, zinc-chloride battery, an alkaline/manganese battery, a silver-oxide battery, a lithium battery, a mercury battery, and a water-activated battery. Furthermore, when the current source is in the form of a water-activated battery, the anode may be selected from a magnesium anode, and a magnesium-zinc alloy, and the cathode maybe selected from the group consisting of silver chloride, cuprous chloride, cuprous bromide, cuprous iodide, and cuprous thiocyanate, copper sulfate, and manganese dioxide.

The alarm device 24 or signal transmitter 26 may be positioned anywhere along the diaper, e.g. attached to the absorbent pad or attached to the outer panel.

While the preferred embodiments of the absorbent article and remote monitoring system have been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. It is to be understood that the invention includes all such variations and modifications which fall within its spirit and scope.

Therefore, the foregoing is considered as illustrative only of the principles of the invention. Further, since numerous modifications and changes will readily occur to those skilled in the art, it is not desired to limit the invention to the exact construction and operation shown and described. It is intended that the invention not be limited to the particular embodiment disclosed as the best mode contemplated for carrying out this invention, but that the invention will include all embodiments falling within the scope of the appended claims.

1. An absorbent article comprising
an outer cover;
an inner absorbent material;
a sensing system, the sensing system comprising:
a measuring device;
a monitoring area having a plurality of conductor pairs being spaced apart a preselected distance and in open circuit configuration positioned in the monitoring area, the conductors being in electrical engagement with the measuring device, at least one conductor pair sensing when an insult has occurred and the size of the insult as the circuit closes; and
a warning device electrically connected to the measuring device, the warning device providing information to the caregiver regarding the occurrence and size of the liquid or solid insult.

2. The absorbent article as recited in claim 1, wherein a power source is electrically connected to the conductors, the power source providing power to the measuring device and the warning device.

3. The absorbent article as recited in claim 2, wherein the conductors have a respective positive and negative charge provided thereon whereby when the absorbent article is dry, a circuit across the conductors is open as an electrical measuring resistance develops between the conductors, and when the absorbent article is soiled, the charge bridges the conductors, completing the circuit.

4. The absorbent article as recited in claim 1, wherein the conductors of the monitoring area have three conductor pairs, a third conductor pair being spaced further apart than a second conductor pair and the second conductor pair being spaced apart further than a first conductor pair.

5. The absorbent article as recited in claim 4, wherein the first conductor pair has a longer length than the second conductor pair and the second conductor pair has a longer length than the third conductor pair.

6. The absorbent article as recited in claim 1, wherein the conductors of the monitoring area form a grid.

7. The absorbent article as recited in claim 2, wherein the warning device is an audible alarm which is activated when a circuit is completed between any respective conductors, the audible alarm will varying depending upon the amount of conductors bridged and the size of the circuit completed.

8. The absorbent article as recited in claim 2, wherein the warning device is a signal transmitter which is activated when a circuit is completed between any respective conductors, a signal transmitted by the signal transmitter will vary depending upon the amount of conductors bridged and the size of the circuit completed.

9. The absorbent article as recited in claim 1, wherein each conductor is capacitively coupled to another respective, when if an insult has occurred, the capacitive coupling of the respective conductors proximate the insult is increased.

10. An absorbent article comprising
an outer cover;
an inner absorbent material;
a sensing system, the sensing system comprising:
a measuring device;
a first monitoring area extending from proximate a front waist region of the absorbent article to proximate a crotch region of the absorbent article, a second monitoring area extending from proximate a back waist region of the absorbent article to proximate the crotch region,
conductors positioned in the first monitoring area and the second monitoring area, the conductors being in electrical engagement with the measuring device, the conductors of the first monitoring area are spaced apart to sense if a liquid insult has occurred and the size of the liquid insult, the conductors of the second monitoring area are spaced apart to sense if a solid insult has occurred and the size of the solid insult; and
a warning device electrically connected to the measuring device, the warning device providing information to the caregiver regarding the occurrence and size of the liquid or solid insult.

11. The absorbent article as recited in claim 9, wherein a power source is electrically connected to the conductors, the power source providing power to the measuring device and the warning device.

12. The absorbent article as recited in claim 10, wherein the conductors have a respective positive and negative charge provided thereon whereby when the absorbent article is dry, a circuit across the conductors is open as an electrical measuring resistance develops between the conductors, and when the absorbent article is soiled, the charge bridges the conductors, completing the circuit.

13. The absorbent article as recited in claim 9, wherein the conductors of the first monitoring area and the second monitoring area each have three conductor pairs, a third conductor pair being spaced further apart than a second conductor pair and the second conductor pair being spaced apart further than a first conductor pair.

14. The absorbent article as recited in claim 12, wherein the first conductor pair has a longer length than the second conductor pair and the second conductor pair has a longer length than the third conductor pair.

15. The absorbent article as recited in claim 9, wherein the conductors of the first monitoring area form a first grid and the conductors of the second monitoring area form a second grid.

16. The absorbent article as recited in claim 10, wherein the warning device is an audible alarm which is activated when a circuit is completed between any respective conductors, the audible alarm will varying depending upon the amount of conductors bridged and the size of the circuit completed.

17. The absorbent article as recited in claim 10 wherein the warning device is a signal transmitter which is activated when a circuit is completed between any respective conductors, a signal transmitted by the signal transmitter will vary depending upon the amount of conductors bridged and the size of the circuit completed.

18. The absorbent article as recited in claim 10, wherein each conductor is capacitively coupled to another respective, when if an insult has occurred, the capacitive coupling of the respective conductors proximate the insult is increased.

19. An absorbent article comprising
an outer cover;
an inner absorbent material;
a sensing system, the sensing system comprising:
a first monitoring area extending from proximate a front waist region of the absorbent article to proximate a crotch region of the absorbent article, a second monitoring area extending from proximate a back waist region of the absorbent article to proximate the crotch region,
a measuring device, the measuring device determining whether a liquid insult has occurred in the first monitoring area and the size of the liquid insult, the measuring device determining whether a solid insult has occurred in the second monitoring area and the size of the solid insult; and
a warning device electrically connected to the measuring device, the warning device providing information to the caregiver regarding the occurrence and size of the liquid or solid insult.

20. The absorbent article as recited in claim 19, wherein a power source is electrically connected to the conductors, the power source providing power to the measuring device and the warning device.

21. The absorbent article as recited in claim 19, wherein the warning device is an audible alarm which is activated when the measuring device determines that an insult has occurred, the audible alarm will varying depending upon the size of the insult as determined by the measuring device.

22. The absorbent article as recited in claim 19 wherein the warning device is a signal transmitter which is activated when the measuring device determines that an insult has occurred, a signal transmitted by the signal transmitter will varying depending upon the size of the insult as determined by the measuring device.
